(19) United States
(12) Patent Application Publication
(10) Pub. No.: US 2026/0345860 A1
(43) Pub. Date: Nov. 5, 2026

(54) ION RELEASING MATERIALS AND USE IN MEDICAL OR DENTAL APPLICATIONS

(71) Applicant: MESOSIL INC., Toronto (CA)

(72) Inventors: Neda ASLANKOOHI, Toronto (CA); Cameron Alexander STEWART, Toronto (CA)

(21) Appl. No.: 19/487,438

(22) PCT Filed: Nov. 22, 2024
(86) PCT No.: PCT/CA2024/051561

§ 371 Date: Nov. 24, 2025

Related U.S. Application Data
(60) Provisional application No. 63/602,534, filed on Nov. 24, 2023.
(60) Provisional application No. 63/570,401, filed on Mar. 27, 2024.

(51) Int. Cl. A61L 27/12; A61K 6/69; A61K 6/76; A61L 27/44; A61L 27/54; A61L 27/56

(52) U.S. Cl. CPC A61L 27/12; A61K 6/69; A61K 6/76; A61L 27/446; A61L 27/54; A61L 27/56; A61L 2300/206; A61L 2300/404

(57) ABSTRACT

A composition has a porous framework containing a resilient oxide and one or more releasable ions. The resilient oxide may be a silicon-oxide or a silicon-phosphorous-oxide. The pores of the framework may be loaded with a biologically active agent. The biologically active agent may be an amphiphilic templating molecule used to form the pores of the framework. The composition may be dispersed in a resin, for example a dental composite or restorative resin. A method of making the composition includes preparing a solution of the biologically active agent so as to form micelles, adding a metal oxide forming compound, and adding an optionally excess amount of a non-metal oxide forming compound and/or one or more metal salts. In a dental application, the active agent may be antimicrobial and the releasable ions may support tooth remineralization.

### RELATED APPLICATION

This application claims the benefit of, and priority from, U.S. Provisional Application No. 63/602,534, filed on Nov. 24, 2023, and U.S. Provisional Application No. 63/570,401, filed on Mar. 27, 2024, each of which are incorporated by reference.

### FIELD

The present disclosure relates to the synthesis and use of ion releasing materials, for example porous metal-oxide or bio-active glass materials, and their application for the release of one or more ions, for example in medical or dental applications.

### BACKGROUND

Amalgam is a metal alloy used to fill tooth cavities made by mixing liquid mercury with particles of solid metals such as silver, copper or tin. Due to the presence of heavy metals, amalgam has been substantially replaced by dental composites in restorative dentistry. Compared to amalgam, dental composites pose less safety concerns and provide better aesthetics.

Dental composites are typically made of methacrylate monomers, a photo-initiator, and a silica filler. Silica-based glasses can be prepared using a melt-quench method or the sol-gel method. The sol-gel method is a low-temperature synthesis reaction that results in glasses or metal oxide materials with higher surface area compared to the melt-quench method. Moreover, the sol-gel method can allow the porosity of a material to be controlled.

One of the drawbacks of dental composites is polymerization shrinkage, which can lead to gaps at the tooth/composite interface. These gaps can permit bacterial penetration which may encourage secondary caries. In some cases, secondary caries can decrease the lifespan of composites compared to amalgam.

Various forms of calcium phosphates, for example, amorphous calcium phosphate (ACP), have been used as fillers in a dental composite. However, ACP is highly soluble, which is detrimental to the mechanical properties of a dental composite compared to silica-based fillers. In some examples, ion-releasing fillers are blended with reinforcing, non-ion-releasing, fillers in the composite.

Antibacterial agents have also been added to dental materials. Examples of antibacterial dental monomers include, but are not limited to, monomers based on quaternary ammonium salts (e.g., polyethylenimine, quaternized thiolene-methacrylate monomers, methacryloyloxydodecyl pyridinium bromide (MDPB) and methacryloxylethyl cetyl dimethyl ammonium chloride (DMAE-CB)). Metal-based antibacterial agents can include zinc oxide nanoparticles or zinc-doped silica nanoparticles, silver nanoparticles, titanium oxide nanoparticles, copper oxide and gold-titanate.

Mesoporous silica or mesoporous metal oxide materials have been investigated as a controlled-release carrier. They are generally prepared using the sol-gel method in the presence of a structure-directing agent as a template. The obtained material undergoes a post-synthesis high-temperature treatment to remove the templating molecules. This results in the generation of open mesopores. For controlled-release applications, the pores are loaded with a cargo of interest after removal of the template. This loading can be through immersion of the material in saturated drug solutions or melted drugs. The size of the drug molecule in comparison to the pore size of the material, and the solubility of the drug in a solvent, are important factors for loading. The drug loading efficiency is normally low and most of the loaded compound resides at the outer surface of the material or in the proximity of pore openings, resulting in a fast or burst release.

International Patent Publication Number, WO 2017/197510 A1, titled “Highly Loaded Metal Oxide Materials by Self-assembly for Extended Biologically Active Molecule Release in Medical and Dental Applications”, describes a biocompatible composite material for controlled release. The material has a metal-oxide structure with a network of pores. The pores are loaded with a biologically active micellizing amphiphilic molecule. This publication is incorporated herein by reference.

### SUMMARY

In one aspect, this specification describes a composition having a porous framework containing a resilient oxide and one or more releasable ions. The resilient oxide may include a metal oxide, for example a silicon-oxide, or a metal/non-metal oxide, for example a silicon-phosphorous-oxide. The releasable ions may be monoatomic ions, for example calcium, phosphorous, magnesium or potassium ions, or polyatomic ions, for example phosphate. The releasable ions may be located within the network, on the surface of the network, or both. The releasable ions are not covalently bound to the network and release, for example into a fluid or in vivo, independently of dissolution of the network.

Optionally, the pores of the framework may be loaded with a biologically active agent. The biologically active agent may be an amphiphilic templating molecule used to form the pores of the framework according to a sol-gel method. Optionally, the composition may be dispersed in a resin, for example a dental composite or restorative resin.

In some embodiments, the composition includes a silica-oxide or silica-phosphorous-oxide network, an antimicrobial agent in the pores of the network, and one or more of calcium, phosphorous, magnesium, potassium and phosphate ions within the network and/or on the surface of the network. The composition may be in the form of particles. In some embodiments, particles of the composition are dispersed in a dental composite or restorative resin.

A method of making a composition includes preparing a solution of a micelle-forming templating agent, optionally a biologically active templating agent. The method also includes, in one or more steps, adding one or more metal oxide forming compounds (typically added in a precursor form, such as an alkoxide), and adding one or more metal salts and/or one or more non-metal oxide forming compounds (typically added in precursor form such as an ester and/or acid). A reaction is allowed to proceed whereby the oxide forming compounds condense into a framework, for example an amorphous glass, around the micellar template. Releasable ions, which may be supplied by the metal salts or by an excess amount of the non-metal oxide forming compounds, may be present within the framework and/or on the surface of the framework. In a dental or medical application, the biologically active agent may be an antimicrobial agent and the releasable ion or ions may support tooth remineralization or hard tissue regeneration.

In one aspect, this specification describes a composition having a porous oxide network. The network (alternatively call a framework or a glass) includes one or more metals (the term metal as used herein including metalloids, for example silicon) bound to oxygen and optionally one or more other (e.g. non-metal) elements such as phosphorous also bound (e.g. covalently bound) to oxygen. The network also contains one or more releasable (e.g. non-covalently bound) elements. The one or more releasable elements may be within the network, on the surface of the network, or both. The one or more releasable elements may be an ion, for example a monoatomic ion such as magnesium, potassium or calcium ions, or a polyatomic ion such as phosphate. Optionally, the pores of the network may contain at least one biologically active agent. In some examples, the biologically active agent is an amphiphilic molecule used as a templating agent to form the network which is present in a self-assembled or micellar form. Optionally, the network is amorphous. Optionally, the composition is mesoporous.

In another aspect, the specification describes a composition having a porous network and one or more biologically active agents in pores of the network, wherein the network comprises one or more releasable elements such as ions. Optionally, the one or more biologically active agents comprise an antimicrobial agent. Optionally, the one or more releasable elements are monoatomic or polyatomic ions comprising one or more of calcium, phosphorous, magnesium and potassium.

This specification also describes a composite material having any composition described herein dispersed in a matrix material. Optionally, the matrix material may be a resin, for example a methacrylate resin or a dental adhesive or restorative (i.e. filling) resin. In the context of a dental restoration, the one or more releasable elements, such as ions, may support re-mineralization of tooth material. A biologically active agent, if present, may inhibit the growth of de-mineralizing bacteria. Optionally, the composition may release the one or more biologically active agents and the one or more releasable elements without dissolution of the network.

In another aspect, this specification also describes a method of making a composition. The method includes preparing a solution of a templating agent, optionally with a catalyst (for example a base), in one or more solvents. One or more precursors of one or more network-forming compounds are added to the solution. The network forming compounds include one or more metal oxides. Optionally, a resulting reaction is allowed to proceed for a first period of time. Optionally, one or more precursors of one or more network-modifying compounds or elements are added to the solution. The network-modifying compounds or elements may include one or more non-metal elements or oxides. Optionally, a network-modifying compound or element may be added in an excess amount to provide one or more releasable ions. Optionally, a resulting reaction is allowed to proceed for a second period of time. Optionally, one or more sources of one or more releasable ions are added to the solution. A resulting reaction is allowed to proceed for a third period of time, for example for a period of time sufficient to form solid particles. The composition (e.g. the solid particles) is then separated from the solvent, and optionally washed and dried. Optionally, a precursor of a network-forming compound may be a metal alkoxide, such as silicon alkoxide. Optionally, a precursor of a network-modifying compound or element may be an acid or ester. Optionally, a source of releasable ions may be a metal salt, optionally a metal-chloride salt, for example calcium chloride, magnesium chloride or potassium chloride. Optionally, a compound may be both a precursor of a network-modifying compound or element and a source of releasable ions. For example, a phosphorous acid or ester such as TEP may supply one or both of a non-metal oxide (e.g. P₂O₅) covalently bound to the framework (i.e. a network-modifying compound) and phosphorous or phosphate ions non-covalently bound to the network (i.e. releasable ions) depending, for example, on the amount added and time of addition to the solution. Optionally, the templating agent is a biologically active agent such as an antimicrobial agent.

In one aspect, this specification describes a composition having a porous oxide network and one or more releasable ions within the network and/or on the surface of the network. Optionally, the network has one or more metals covalently bound to oxygen. Optionally, the one or more metals includes a metalloid, for example silicon. Optionally, the network further has one or more non-metals bound to the oxygen. Optionally, the one or more non-metals comprises phosphorous. Optionally, the network comprises a silica-phosphorous-oxide. Optionally, the one or more releasable ions are non-covalently bound to the network. Optionally, at least some of the one or more releasable ions are within and/or beneath the surface of the network. Optionally, at least some of the one or more releasable ions are on the surface of the network. Optionally, the one or more releasable ions comprise a polyatomic ion, for example phosphate. Optionally, the one or more releasable ions comprise calcium, phosphorous, magnesium or potassium. Optionally, the composition releases the one or more releasable ions without dissolution of the network. Optionally, pores of the network contain one or more biologically active agents. Optionally, the one or more biologically active agents include an amphiphilic templating agent present in a self-assembled or micellar form. Optionally, the composition releases the one or more biologically active agents without dissolution of the network. Optionally, the one or more biologically active agents include an antimicrobial agent. Optionally, the biologically active agent is any one of octenidine dihydrochloride, polyhexamethylene biguanide, cetylpyridinium chloride, lauric arginate, and Benzalkonium chloride. Optionally, the biologically active agent content in the composition is in the range of 20-60 wt %. Optionally, the network is amorphous. Optionally, the network is mesoporous. Optionally, the composition is dispersed in a matrix material. Optionally, the matrix material is a polymeric resin. Optionally, the matrix material is a dental adhesive or restorative resin. Optionally, the one or more releasable ions supports the re-mineralization of tooth material. Optionally, the composition comprises an antimicrobial agent. Optionally, the composition is formulated for use as a dental material, an orthopedic implant or bone cement material, a 3D printing ink or filament material, a cosmetic material, or an agricultural material. Optionally, the surface of the composition is modified to bear organic functional groups. Optionally, the composition forms chemical bonds with the matrix material. Optionally, the composition is incorporated into the matric material by contra-centrifugal mixing, orbital mixer, shear mixer, sonication, and stirring with the polymer resin components. Optionally, the composition includes organic additives, such as polymerization inhibitors or color stabilizers, and/or inorganic additives such as fillers.

In one aspect, the specification describes a composition having a porous network, one or more biologically active agents in pores of the porous network, and one or more releasable ions. Optionally, the one or more biologically active agents includes an antimicrobial agent. Optionally, the one or more releasable ions are monoatomic or polyatomic ions including one or more of calcium, phosphorous, magnesium and potassium. Optionally, the porous network has a compound of oxygen and silica or a compound of oxygen, silica and phosphorous. Optionally, the biologically active agent is an anti-microbial agent. Optionally, the biologically active agent is any one of octenidine dihydrochloride, polyhexamethylene biguanide, cetylpyridinium chloride, lauric arginate, and Benzalkonium chloride. Optionally, the biologically active agent content in the composition is in the range of 20-60 wt %. Optionally, the composition is dispersed in a matrix material. Optionally, the matrix material is a polymeric resin. Optionally, the matrix material is a dental adhesive or restorative resin. Optionally, the one or more releasable ions supports the re-mineralization of tooth material. Optionally, the composition comprises an antimicrobial agent. Optionally, the composition is formulated for use as a dental material, an orthopedic implant or bone cement material, a 3D printing ink or filament material, a cosmetic material, or an agricultural material. Optionally, the surface of the composition is modified to bear organic functional groups. Optionally, the composition forms chemical bonds with the matrix material. Optionally, the composition is incorporated into the matric material by contra-centrifugal mixing, orbital mixer, shear mixer, sonication, and stirring with the polymer resin components. Optionally, the composition includes organic additives, such as polymerization inhibitors or color stabilizers, and/or inorganic additives such as fillers.

In one aspect, the specification describes a method of making a composition having steps of preparing a solution of a micelle-forming templating agent; adding to the solution, in one or more steps, a) one or more metal oxide forming compounds and b) one or more metal salts; and, allowing the reaction to proceed whereby a metal oxide condenses into a network around the templating agent. Optionally, the one or more metal salts are added to the solution at least 1 minute, at least 3 minutes or at least 5 minutes after adding the one or more metal oxide forming compounds to the solution. Optionally. the one or more metal oxide forming compounds comprise a metal alkoxide. Optionally, the metal alkoxide is silicon alkoxide. Optionally, the one or more metal salts comprise calcium, potassium or magnesium. Optionally, the one or more metal salts comprise a chloride salt. Optionally, the method includes adding one or more non-metal oxide forming compounds to the solution. Optionally, the one or more non-metal oxide forming compounds includes an ester and/or acid. Optionally, the one or more non-metal oxide forming compounds comprise phosphorous. Optionally, the network includes a non-metal oxide such as a phosphorous oxide, for example wherein the framework comprises a metal/non-metal oxide such as silica-phosphorous-oxide. Optionally, the network includes an amorphous glass. Optionally, the network comprises releasable ions. Optionally, the releasable ions are present within the framework. Optionally, the releasable ions comprise calcium and phosphate. Optionally, the templating agent is a biologically active templating agent. Optionally, the templating agent is an antimicrobial agent, for example OCT.

In one aspect, this specification describes a method of making a composition including steps of preparing a solution of one or more templating agents, optionally with a catalyst, in one or more solvents; adding one or more precursors of one or more network-forming compounds to the solution; optionally allowing a resulting reaction to proceed for a first period of time; optionally adding one or more precursors of one or more network-modifier compounds to the solution; optionally allowing a resulting reaction to proceed for a second period of time; optionally adding one or more sources of releasable ions; allowing a resulting reaction to proceed for a third period of time; and, separating the composition from the solvent. Optionally, the network forming compounds include one or more metal oxides. Optionally, the network-modifier compounds include one or more non-metal oxides. Optionally, the releasable ions include one or more metal ions or polyatomic ions. Optionally, the one or more precursors of the one or more network-forming compounds includes a metal alkoxide, such as silicon alkoxide. Optionally, the one or more sources of releasable ions includes one or more metals salt. Optionally, the one or more metal salts comprises a metal-chloride salt. Optionally, the one or more metal salts includes calcium chloride, magnesium chloride and/or potassium chloride. Optionally, the one or more precursors of the one or more network-modifier compounds comprises a phosphorous acid or ester, such as TEP. Optionally, the templating agent is a biologically active agent such as an antimicrobial agent. Optionally, the method includes washing the composition and drying the composition. Optionally, the one or more sources of releasable ions includes a salt of one or more of calcium, boron, zinc, magnesium, manganese, copper and potassium. Optionally, the biologically active agent is any one of octenidine dihydrochloride, polyhexamethylene biguanide, cetylpyridinium chloride, lauric arginate, and benzalkonium chloride.

### BRIEF DESCRIPTION OF THE DRAWINGS

The description of drawings accompanying this disclosure is given below:

FIG. 1A is a schematic diagram, not to scale, showing a first method of synthesizing a mesoporous particle having a biologically active agent loaded after forming pores.

FIG. 1B is a schematic diagram, not to scale, showing a method of synthesizing a mesoporous particle having a biologically active templating agent.

FIG. 2 is a flowchart showing steps in a method for making a composition having a porous (e.g. mesoporous) oxide (e.g. metal oxide or metal/non-metal oxide) network templated on an amphiphilic, optionally biologically active, agent and having one or more releasable elements (e.g. monoatomic or polyatomic ions).

FIG. 3A shows schematically and not to scale a composition having an amphiphilic biologically active templating agent within the pores of a metal/non-metal oxide network (in this example a silica and phosphorous oxide network) with releasable elements (in this example calcium and phosphorous and/or phosphate ions) within the network but not covalently bound to the network.

FIG. 3B shows schematically and not to scale a composition having an amphiphilic biologically active templating agent within the pores of a primarily metal oxide network (in this example a silica oxide network, optionally with some covalently bound phosphorous near the surface) with releasable elements (in this example calcium and phosphorous and/or phosphate ions) on the surface of the network but not covalently bound to the network.

FIG. 4 shows transmission electron microscope (TEM) images of particles with different compositions.

FIG. 5 shows a scanning electron microscope (SEM) image of particles and elemental mapping by energy dispersive X-ray analysis (EDX).

FIG. 6A-6C are charts showing the release of ions from the particles with different compositions.

FIG. 7 is a chart showing the release profile of an amphiphilic biologically active agent, octenidine dihydrochloride (OCT), at 37 °C in PBS from particles with similar nominal compositions but different oxide(s) structure.

FIG. 8 is a chart showing the release profile of the amphiphilic biologically active agent, octenidine dihydrochloride, at 37 °C in PBS from particles with similar nominal compositions but different silicon content.

FIG. 9 is a chart showing the release profile of the amphiphilic biologically active agent, octenidine dihydrochloride, at 37 °C in PBS from particles having different binary or ternary compositions.

FIG. 10 is a chart showing the release profile of the amphiphilic biologically active agent, octenidine dihydrochloride, at 37 °C in PBS from particles with the same framework composition but different OCT loadings.

FIG. 11 show TEM images of particles of two exemplary compositions after incubation at 37 °C in PBS for 33 days.

FIG. 12 is a chart showing the degree of conversion (DC) of methacrylate-based polymeric matrices containing various contents of particles with a ternary composition (SiO₂—CaCl₂—P₂O₅) compared to the polymeric matrix without the particles.

FIG. 13 is a chart showing S. mutans biofilm growth on methacrylate-based polymeric matrices containing 2% and 5% of particles of the composition with a ternary composition (SiO₂—CaCl₂—P₂O₅) compared to the polymeric matrix without the particles.

### DETAILED DESCRIPTION

Various apparatuses, compositions or processes will be described below to provide an example or an embodiment of each claimed invention. No example or embodiment described below limits any claimed invention and any claimed invention may cover processes, compostions or apparatuses that differ from those described below. The claimed inventions are not limited to apparatuses, compositions or processes having all of the features of any one apparatus, composition or process described below or to features common to multiple or all of the apparatuses, compositions or processes described below. It is possible that an apparatus, composition or process described below is not an embodiment of any claimed invention. Any invention disclosed in an apparatus, composition or process described below that is not claimed in this document may be the subject matter of another protective instrument, for example, a continuing patent application, and the applicants, inventors or owners do not intend to abandon, disclaim or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention of being bound by any expressed or implied theory presented in the preceding technical field, background, brief summary or the following detailed description.

As used herein, the wording “and/or” is intended to represent an inclusive-or. That is, “X and/or Y” is intended to mean X or Y or both X and Y, for example. As a further example, “X, Y, and/or Z” is intended to mean X or Y or Z or any combination thereof.

Terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as by 1%, 2%, 5%, or 10%, for example.

Reference throughout this specification to “one embodiment”, “an embodiment”, “at least one embodiment” or “some embodiments”, or similar references to example(s), means that any one or more features, steps, structures, or characteristics describe in relation to any one or more embodiments may be combined in any suitable manner with any one or more features, steps, structures, or characteristics describe in relation to any other one or more embodiments, unless specified to be not combinable or to be strictly alternative options.

Various aspects of the disclosure are described herein with reference to the embodied figures. The aforementioned figures are presented exemplarily and are not intended to limit the disclosure to certain release patterns, morphology, particle sizes (for nanoparticle morphology), oxide(s) structure composition, etc.

The term ‘oxides’ refers to metal (including metalloid) oxides and optionally to molecules combining one or more metals and one or more non-metals with oxygen. The ‘oxides’ may have some unreacted and/or bridging organic groups and/or reactive functional groups.

The term ‘biologically active agent’ refers to any natural or synthetic compound for preventing or treating a disease.

As used herein, the term ‘amphiphilic’ refers to molecules having at least two groups with different solvent interaction properties. An amphiphilic molecule is preferably associated with a specific characteristic concentration named ‘critical micelle concentration (CMC)’ above which the amphiphilic molecules self-assemble to form micelles in solution.

As used herein, metal oxides include metalloid oxides such as silica oxides. The metal oxides are typically in an amorphous, networked or glass form. The term metal oxides does not preclude the presence of other elements or oxides. A metal/non-metal oxide may include a metal, such as a silica, and a non-metal, such as phosphorous or phosphates, covalently bound to oxygen atoms of the oxide.

This specification describes a composition, typically in the form of particles but optionally a coating, having a porous framework. The framework includes a resilient oxide including one or more metals, such as silica, and optionally one or more non-metals, such as phosphorous, covalently bound to oxygen. The framework also contains one or more releasable, i.e. non-covalently bound, ions. The releasable ions may be located within the framework and/or on the surface of the framework. In some embodiments, the releasable ions are one or more of magnesium, potassium, calcium, phosphorous and phosphate. Optionally, the pores of the framework may be loaded with at least one biologically active agent, for example an antimicrobial agent. In some embodiments, the biologically active agent is a micellizing amphiphilic molecule used as a templating agent during formation of the framework.

A method of making a composition includes preparing a solution of a templating agent to form micelles, adding a metal oxide forming compound to the solution, and adding one or more metal salts and/or non-metal oxide forming compounds to the solution.

In some embodiments, an amphiphilic biologically active agent is used both as a template as well as the cargo of interest and the ion(s) of interest are incorporated during synthesis of the oxide structure. Therefore, the synthesis can optionally be conducted in one step, without the need for any post-synthesis treatment to incorporate ions or load a biologically active agent. The sol-gel technique derivative used here results in incorporation of the biologically active agent in the oxide structure during the synthesis without negatively affecting the activity of the agent. The release of the biologically active agent is driven mainly by diffusion as opposed to by dissolution of the oxide framework.

Depending on the incorporated ion(s), and the biologically active agent, a composition can fulfil requirements for different applications. For instance, a binary or ternary composition containing calcium and/or phosphorus (optionally referred to in some examples as SiO₂—CaCl₂—P₂O₅, SiO₂—CaCl₂ or SiO₂—P₂O₅), loaded and optionally templated with an antimicrobial compound such as octenidine dihydrochloride, can be used in applications directed to hard tissue. Release of calcium and phosphate ions can promote formation of hydroxyapatite (the inorganic phase of hard tissue), and hard tissue remineralization/regeneration. Release of the antimicrobial agent octenidine can reduce bacteria growth adjacent to the material.

Optionally, a composition is prepared in the form of particles and incorporated in a dental composite resin or adhesive.

Dental enamel and dentin demineralization and remineralization processes occur repeatedly in an individual's lifetime. In dental caries, demineralization is promoted by acids produced by biofilm bacteria and overbalances remineralization. Caries can be arrested in the early stages by disturbing the bacteria and promoting the remineralization of dentin. Dental restorations that can inhibit bacterial growth and simultaneously promote remineralization by the release of ions such as calcium and phosphate can inhibit secondary caries.

A exemplary composition described herein can provide a multi-functional filler that releases both remineralizing ions as well as an antibacterial agent from a single filler component. As such, the composition can be used to inhibit secondary caries and/or to extend the lifespan of a dental restoration. The composition can be used as a filler in a dental composite resin or alternatively in an adhesive at the dentin-restoration interface, in a cement, in a root canal sealer, in an implant coating or in other applications in the oral cavity.

Silica-based glasses have been long used as fillers in restorative composites for dentistry. Therefore, a multi-functional silica-based filler as described herein can be incorporated into a typical clinical workflow of preparing dental composites or other materials.

Beyond dentistry, a composition as described herein can be used in various fields of medicine. For example, in the field of orthopedics, the composition may be used as a filler in a bone cement, as a component of a coating on an implant surface, or in other applications where the release of an antimicrobial agent and bio-relevant ions may be beneficial and/or therapeutic, for example through the prevention of infection and the promotion of bone remodeling and growth. In other examples, the composition may have uses in food as a preservative and source of dietary ions, or as an ion source and pesticide/fungicide/bactericide for fertilizers, or in certain chemical processes requiring simultaneous release of small bioactive molecules and ions.

Ion-releasing materials have been used by the medical field due to the biological effects of ions. For example, magnesium ions can show both antibacterial and osteogenic effects. Calcium and phosphate ions can be used as contributors to (re)mineralization of hard tissues (i.e., bone and teeth), and as building blocks of hydroxyapatite. The co-delivery of ions and biologically active agents can provide synergistic effects and improve therapeutic outcomes. The ions may be released into a fluid, for example water, a buffer, interstitial fluid, blood or saliva, or in vivo, for example in the mouth or body of a person.

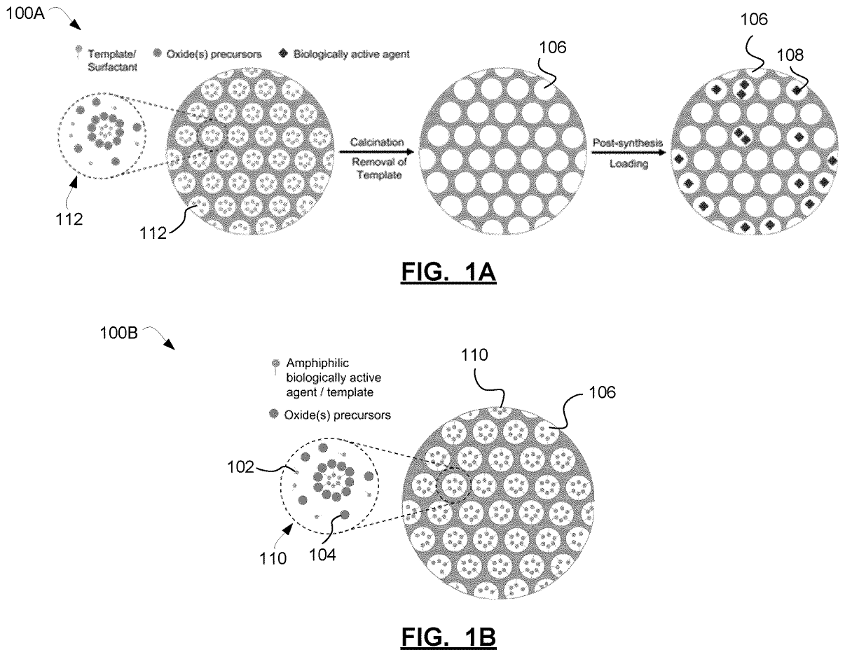

Turning now to the figures, FIG. 1A provides a schematic diagram showing a method of synthesizing a porous framework, for example in the form of a particle, according to a first method 100A.

In the first method 100A of preparing the framework, a templating agent 112, for example a surfactant, is prepared in a solution and forms micelles. An oxide precursor is added to the solution and condenses around the templating agent 112 forming particles. The particles are separated from the solution. The particles are calcined to remove the templating agent 112 leaving open pores 106 in an oxide framework. A biologically active agent 108 may then be loaded in the pores. However, given the tortuous pore network 106, loading the biologically active agent 108 is diffusion-limited and most of the biologically active agent 108 typically resides on the surface of the material or close to the pore openings. This surface-biased loading may result in a fast, or burst, release profile. The biologically active agent 108 typically does not completely fill the pore volume. This method permits producing compositions wherein the biologically active agent 108 does not have the qualities of a templating agent. However, the hydrodynamic size of the biologically active agent should ideally be close to the pore diameter to increase the loading of the biologically active agent 108 in the pores by diffusion and adsorption.

FIG. 1B provides a schematic diagram showing a method 100B of synthesizing a porous network according to the method described in International Patent Publication Number, WO 2017/197510 A1, titled “Highly Loaded Metal Oxide Materials by Self-assembly for Extended Biologically Active Molecule Release in Medical and Dental Applications”, which is incorporated by reference herein. In the synthesis method 100B, an amphiphilic molecule 102 is used as both the template and the biologically active agent. The amphiphilic molecule 102 is present above its critical micelle concentration (CMC), such that the amphiphilic biologically active agent self-assembles to form micelles 110. In method 100B, the entire volume of pores 106 is filled with the amphiphilic molecule 102, which is also the biologically active agent.

The interconnected network of pores extends throughout the oxide structure, reaching the structure's surface. The biologically active agent releases from pores by diffusion, upon contact with a medium such as PBS or saliva. The oxide network forms by hydrolysis and condensation and/or assembly/interaction of its precursors. The amorphous oxide species assemble around the micelles of the biologically active agent. These micelles form an interconnected network of pores within the oxide(s) structure.

The diameter of the pores is dependent on the size of the biologically active agent's micelles. The use of a hydrophobic or a hydrophilic biologically active agent in the core of the amphiphilic biologically active agent micelles results in the increase of micelle size and subsequently increase of pore diameter. The total porosity volume (i.e., total biologically active agent content) is dependent on the synthesis conditions.

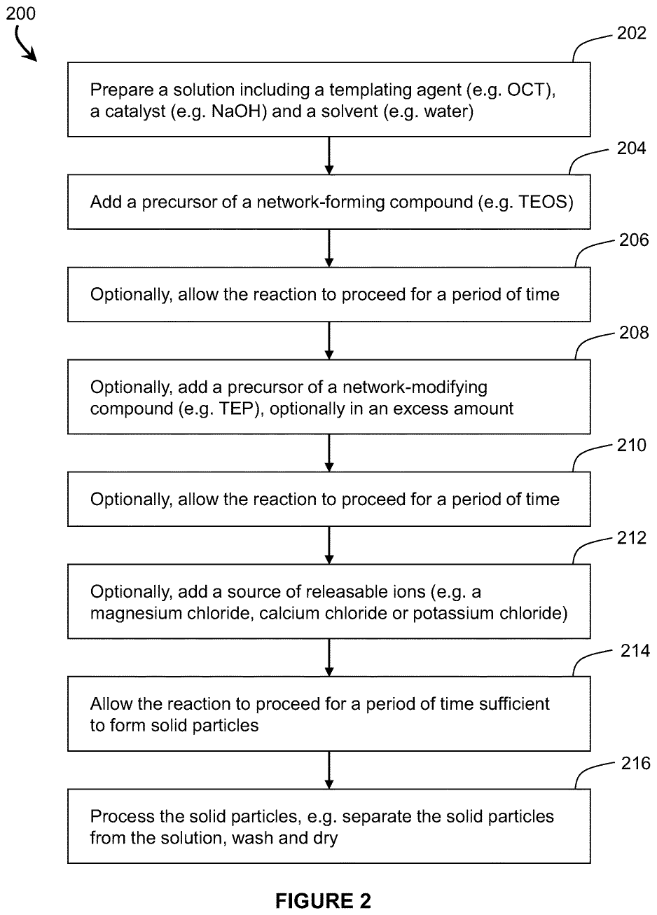

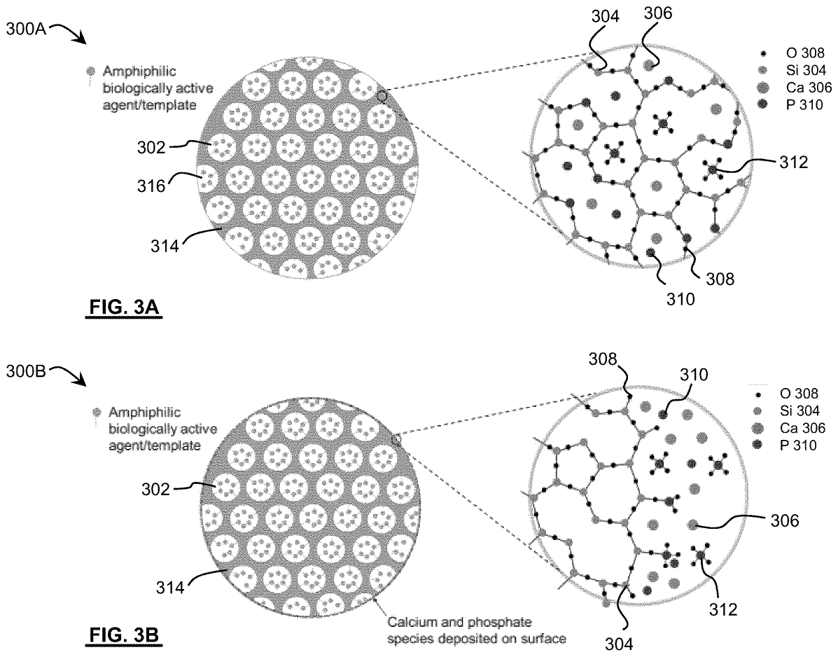

FIG. 2 provides a flowchart of a method 200 for the synthesis of a composition having releasable ions. In some examples, the composition is a biocompatible mesoporous material containing an amphiphilic biologically active agent in a metal oxide or metal/non-metal oxide(s) structure. The disclosed synthesis procedure is versatile in terms of morphology, particle size (for nanoparticle morphology), oxide(s) composition, the type of biologically active agent and the type of releasable ions.

In an embodiment, the method 200 of synthesis can include: preparing a solution of a templating agent (optionally a biologically active agent), a (base) catalyst and a solvent(s) at step 202; adding one or more precursors of one or more network-forming compounds such as a silicon alkoxide (e.g. tetraethyl orthosilicate (TEOS)) at step 204; optionally allowing a reaction to proceed for a first period of time at step 206, the period of time, if any, selected depending on the desired location of any network-modifiers within the structure; optionally adding one or more precursors of one or more network-modifying compounds (the term network-modifying compounds optionally including network-modifying elements) of interest, for example tetraethyl phosphate (TEP), at step 208; optionally allowing the reaction to proceed for a second period of time at step 210, the second period of time selected depending on the desired location of releasable ions within the structure; optionally adding one or more sources of releasable ions at step 212; allowing the reaction to proceed for a period of time sufficient to form a solid material, e.g. particles, at step 214; and, processing the solid material at step 216, for example separating the obtained solid material, washing, for example with water, and drying the material, for example by heating at 60 °C.

In at least some embodiments, the one or more precursors of one or more network-modifying compounds in step 208 provide compounds or elements that become covalently bound in the network. Optionally, the one or more precursors of one or more network-modifying compounds in step 208 may also provide releasable ions that are not covalently bound to the composition. In at least some embodiments, the one or more sources of releasable ions added in steps 212 primarily provide ions that are not covalently bound to the network though they may affect one or more physical attributes of the network. In at least some embodiments, one or both of steps 208 and 212 are present. Depending on the presence and length of steps 206 and/or 210, the formation of a solid material may have commenced before step 214, which may be interpreted as a period of time sufficient to complete the formation of a solid material. For brevity, terms such a network former may refer to a network-forming compound or its precursor as appropriate in the context of where the term is used. For brevity, terms such as network modifier may refer to a network-modifying compound or its precursor as appropriate in the context of where the term is used.

In at least one embodiment, the templating agent, for example an amphiphilic biologically active agent, is dissolved in the solvent in a concentration of at least its CMC. The network forms by hydrolysis, condensation and/or assembly and/or precipitation/deposition of oxide precursors such as but not limited to alkoxides, esters, acids and salts. In at least some embodiments, network former oxide(s) (such as silica) are introduced into the reaction first. Aggregation and growth of metal oxide(s) around the self-assembled micelles of the biologically active agent results in the formation of a solid, optionally biocompatible, material. Sequentially, introducing a network modifier results in the incorporation of one or more additional elements or compounds in the growing metal oxide network. The timing of introducing the network modifier affects their incorporation rate as well as the morphology of the final material. Optionally, adding the network modifier directly after the network former may result in the network modifier being present throughout, or at least in a substantial part of, the framework. Alternatively, adding the network modifier after a period of time may result in the network modifier being present mostly near or on the surface of the framework. Varying the amount of the network modifier can increase or decrease the amount of any releasable ions that are created by the network modifier. An excess amount of the network modifier may be any amount that produces releasable ions. Similarly, the timing of introducing a source of releasable ions can affect the location of these releasable ions in the framework. Optionally, apart from the main network former oxide(s), other compounds can be introduced towards the end of the reaction to form a secondary shell on the surface of the material, with the point in time of addition controlling where in the final material the new compound is included.

The oxide(s) structure may contain hydroxyl groups. The hydroxyl groups which are not sterically hindered can be used as focal points for chemical modification post-synthesis. The modification can be through the covalent attachment of organic functional groups or with different types of (organo)alkoxides that undergo hydrolysis and condensation to form a shell around the previously formed core.

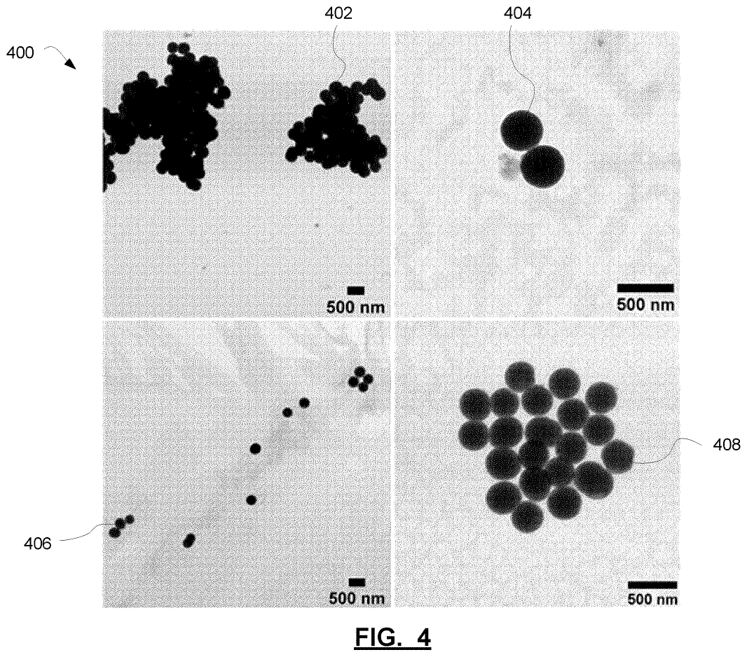

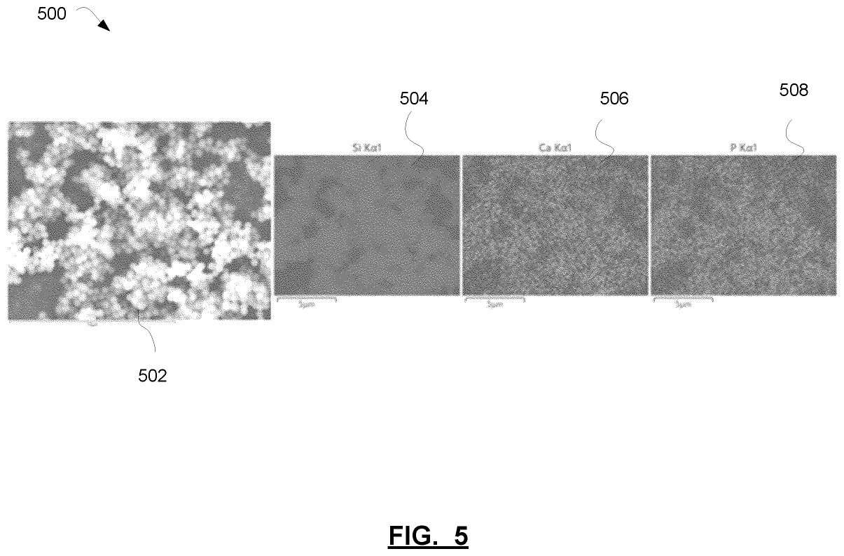

FIG. 3A shows schematically a first material 300A having a mesoporous oxide structure 314 with an amphiphilic biologically active agent 302 templating and filling the pore network 316. The material 300A includes silica 304, calcium 306, oxygen 308, phosphorus 310 and phosphate 312. In at least one embodiment, the material 300A provides an ion and molecule delivery system having a mesoporous material 314 with a framework of silica-and/or other metal-oxide, which may contain pores 316 filled with a releasable optionally templating biologically active agent (such as an amphiphilic antimicrobial agent), and whose silica and/or other metal-oxide framework is partially formed by (covalently linked to) one or more non-metal elements and has releasable (e.g. non-covalently bound) ions. The material 300A can have an amphiphilic biologically active agent 302 within the porosity of a silica matrix (alternatively called a framework, network or glass). The material 300A can have releasable ions such as calcium, phosphorous and/or phosphate ions. The material 300A can also contain phosphorous within the silica matrix, wherein the phosphorous is covalently bound.

FIG. 3A further provides, as an exemplary composition, a ternary Si—Ca—P particle. Here, Si oxides and optionally P oxides 310 are network formers/modifiers and covalently linked, while Ca ions 306 and optionally some P 310 ions or P-containing ions (PO₄) 312 are releasable ions associated with the particles. Not depicted are unreacted organic groups from the silica-precursor molecules (silica alkoxides) which may be present. The presented schematic of FIG. 3A shows some of the possible locations for the releasable ions.

Alternatively or additionally, the releasable ions can be deposited on the surface of a material, optionally forming a secondary shell, as shown for a second material 300B shown schematically in FIG. 3B. The timing of introducing the precursors of the network-modifying compound (e.g. non-metal oxide), the type of precursors for each oxide species, and the timing of introducing any sources of other releasable ions are some of the parameters that may determining the location of releasable ions in the structure of the material. The positioning of ion(s) within the final material can determine when and how the ion(s) are released, providing control over the release kinetics (e.g. immediately from a shell as in FIG. 3B, or over a longer period of time when incorporated into the center or metal oxide region of the material as in FIG. 3A).

The mesoporous material 300A/B can deliver different ion(s), as well as different biologically active agent(s) to elicit (an) effect(s), such as an antimicrobial effect and a dental remineralizing effect simultaneously from a single material. In some examples, an amphiphilic biologically active agent is used both as a template as well as the cargo of interest and the ion(s) of interest are simultaneously incorporated during synthesis of the material 300A/B. Therefore, the synthesis could be conducted in one step, i.e. without the need for any post-synthesis treatment to incorporate ions or load a biologically active agent. The sol-gel technique derivative used here results in incorporation of the biologically active agent in the oxide structure during the synthesis without negatively affecting the activity of the agent. In some embodiments, the release of the biologically active agent is driven mainly by diffusion as opposed to by dissolution of the oxide structure. However, depending on the compositions of oxide(s) structure and subsequently the degradability/resorption rate of the metal-oxide structure, the release can be modulated.

Depending on the incorporated ion(s), and the biologically active agent, the biocompatible material can fulfil requirements for different applications. For instance, a binary or ternary composition having a framework oxide structure and containing calcium and/or phosphorus (optionally referred to in some examples SiO₂—CaCl₂—P₂O₅, SiO₂—CaCl₂ or SiO₂—P₂O₅), loaded and optionally templated with an antimicrobial compound such as octenidine dihydrochloride (OCT), can be used in applications directed to hard tissue. Release of calcium and phosphate ions can promote formation of hydroxyapatite (the inorganic phase of hard tissue), and hard tissue remineralization/regeneration. Release of the antimicrobial agent octenidine can reduce bacteria growth adjacent to the material.

In at least one embodiment, the oxide(s) structure may contain hydroxyl groups. The hydroxyl groups which are not sterically hindered can be used as focal points for chemical modification post-synthesis. The modification can be through the covalent attachment of organic functional groups or with different types of (organo)alkoxides that undergo hydrolysis and condensation to form a shell around the previously formed core.

In at least one embodiment, the oxides uniformly constitute the framework, while the active agent is self-assembled within the framework. The material may release the active agent in a sustained manner, and the composition of the oxide(s) framework may be one of the means to control the release rate.

In at least one embodiment, the composition has an amorphous framework containing one or more oxide species, a biologically active agent in the pores of the framework, and one or more releasable ions associated with the framework. The composition may release the biologically active agent. The composition may also release the ion(s) without dissolution of the framework, for example in the manner of a bio-active glass. Optionally, the pore structure of the framework is produced by formation of the structure around an assembly of the biologically active agent molecules.

In at least one embodiment, a material has a porous metal oxide structure, a releasable templating agent in the porous metal oxide structure, and a releasable ion that has chemical or biological activity associated with the metal oxide structure. The material may be dispersed in a resin, for example a dental composite or restorative resin. In some examples, the releasable ion is tooth remineralizing ion.

FIG. 4 provides transmission electron microscope (TEM) images 400 of biocompatible material in the form of particles with different compositions. In the examples shown in FIG. 4, biocompatible material in the form of spherical particles synthesized by octenidine dihydrochloride (OCT) is shown as an exemplary use of an amphiphilic biologically active agent with different oxide(s) structures. The particles labelled 402 and 404 in FIG. 4 are the same particles but shown at different magnifications. Particles 402, 404 were made according to Example 1 described further below. Particles labelled 406 and 408 in FIG. 4 are the same particles but shown at different magnifications. Particles 406, 408 were made according to Example 3 described further below.

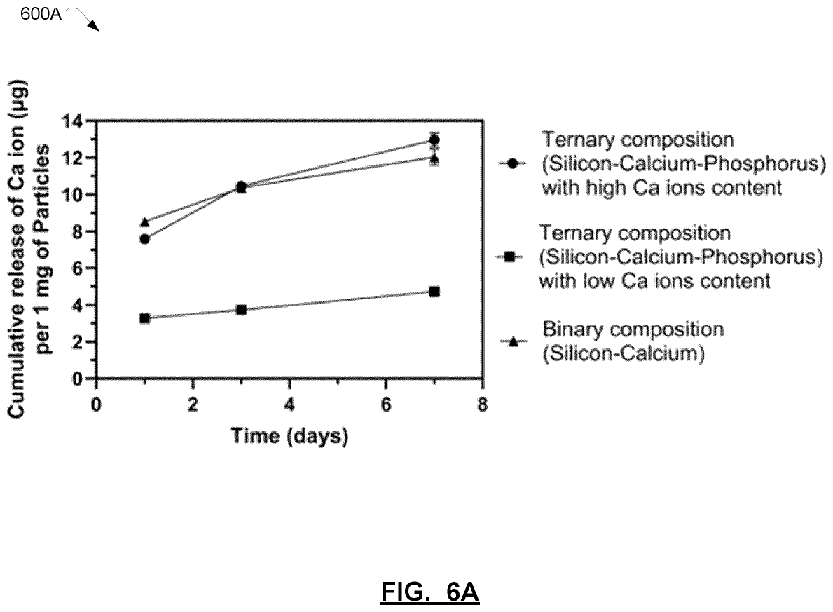

FIG. 5 shows exemplary SEM-EDX images 500 of a composition (in the form of spherical nanoparticles) consisting of OCT and a ternary SiO₂—CaCl₂—P₂O₅ composition 502. The ternary composition has a nominal molar composition of 95.3% silica, 3.97% calcium and 2.54% phosphorous. The uniform distribution of silicon 504, calcium 506, and phosphorus 508 in the particles represents the versatility of the oxide(s) composition, the controllability of the reaction and the homogeneity of the final material.

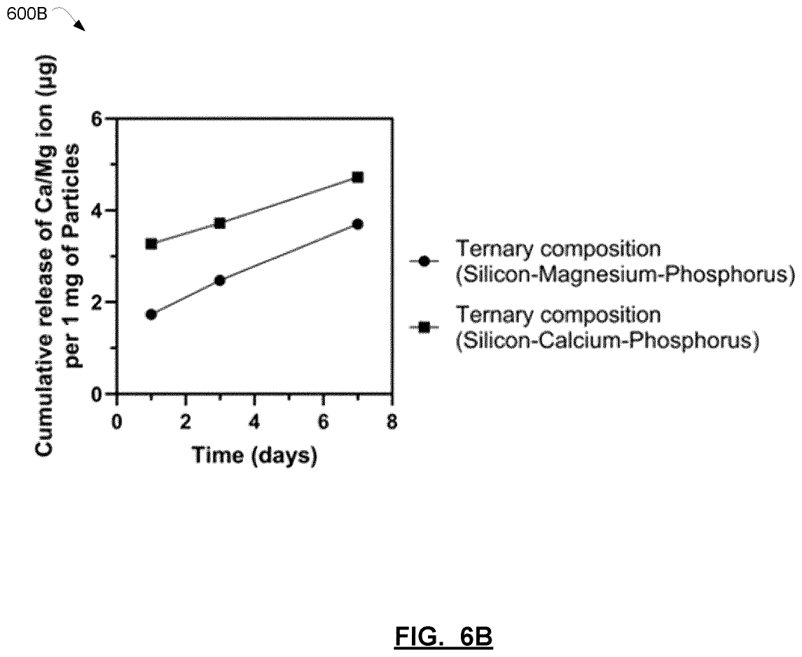

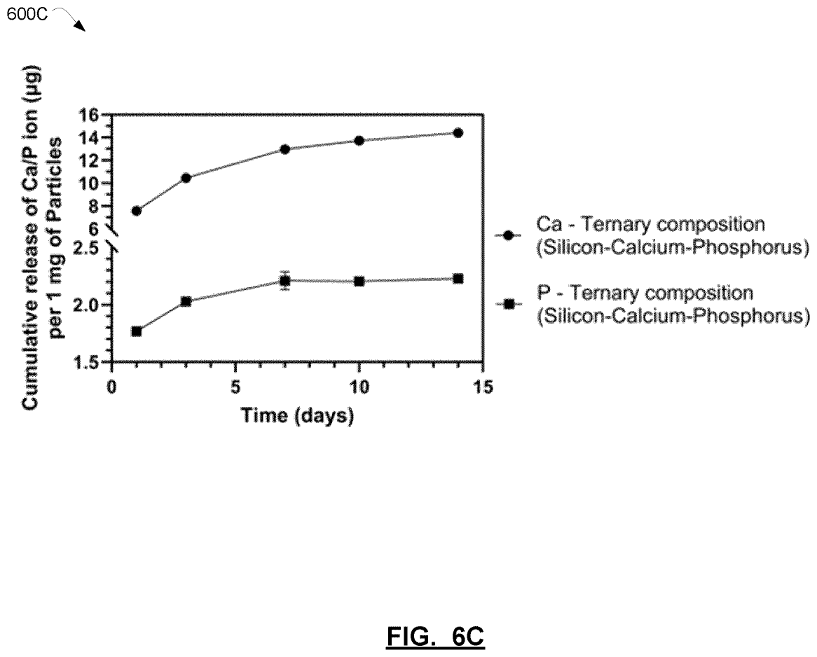

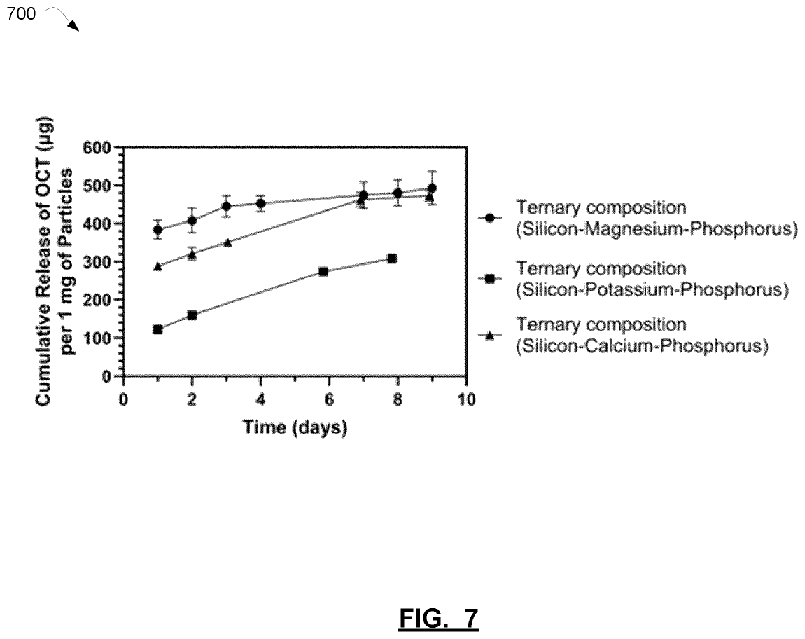

FIGS. 6A-C provides a series of charts 600A, 600B and 600C showing the rate of release of calcium, phosphate, and magnesium ions from biocompatible material in the form of spherical particles with different oxide(s) compositions immersed in PBS at 37 °C with constant gentle shaking. Increasing the amount of initial calcium and phosphate precursors directly resulted in an increase in incorporation of the ions in the biocompatible material and a subsequent increase in released ions. Moreover, the release of phosphate ions did not affect the release rate of calcium ions considerably for materials with the same calcium content (FIG. 6A). For materials with the same nominal composition but with different ions, the ion release rate is different (FIG. 6B). Therefore, the type of ion(s) and the type of precursor can affect the release rate of ions. FIG. 6C shows that in a material with a ternary composition, the release of calcium and phosphate ions followed a similar trend and was consistent with their initial content in the material. The cumulative release approached a plateau after about two weeks. However, incorporation of the material in a polymeric matrix such as a dental resin composite would increase the time length of the release.

In FIGS. 6A and 6B, the cumulative release of the ions from multiple materials was determined by measuring the release of each ion, and adding the results for the different ions together. In FIG. 6C the release of calcium and phosphorous ions from the same material is shown separately.

The rate of release of the biologically active agent can be modified by changing the composition of the oxide(s) structure. The oxide(s) structure can be less compact and/or more hydrophilic to modulate the release rate of the biologically active agent. Depending on the composition of the oxide(s) structure, the integrity of the structure differs. This integrity is not only a tool to modulate the release rate of the biologically active agent but also provides the opportunity for other biological functionalities through the deliberate introduction and release of ions from the structure and fulfilling application-specific requirements in terms of degradability or stability.

In compositions designed for a high release rate of ions, post-synthesis loading of the biologically active agent, as is the case of the method shown in FIG. 1A, is not possible due to the loss of a considerable amount of ions during the loading process. Therefore, the disclosed synthesis method expands the range of possible practical compositions having an oxide(s) structure.

Surface area (or particle size in case of biocompatible material with spherical particle morphology) is another factor that can be used to modulate the release profile of the biologically active agent.

FIGS. 7 to 10 provide a series of charts showing the release profile of OCT from particles with different compositions. These figures demonstrate the ability to modulate the release rate by changing the composition of the particle, as well as changing the biologically active agent loading by adjusting the synthesis parameters.

FIG. 7 provides a chart 700 showing the release profile of OCT from three ternary compositions of SiO₂—XCl₍₂₎—P₂O₅ where X is a counterion to Cl. A significant difference between the release rates can be seen on initial days, after which the release rate of compositions containing magnesium and calcium converged to be similar while the composition containing potassium had a significantly lower release. Under similar synthesis conditions, the total OCT loading was higher in the presence of divalent cations.

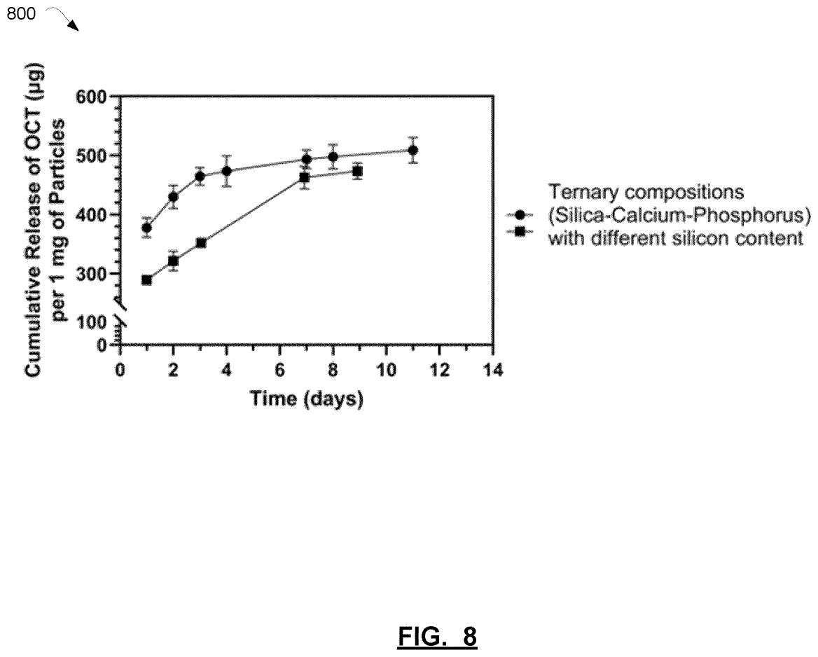

FIG. 8 provides a chart 800 showing the release profile of OCT from two ternary compositions of SiO₂—CaCl₂—P₂O₅. Again, a significant difference between the release rates can be seen on initial days. The composition with lower silica content showed a higher release rate.

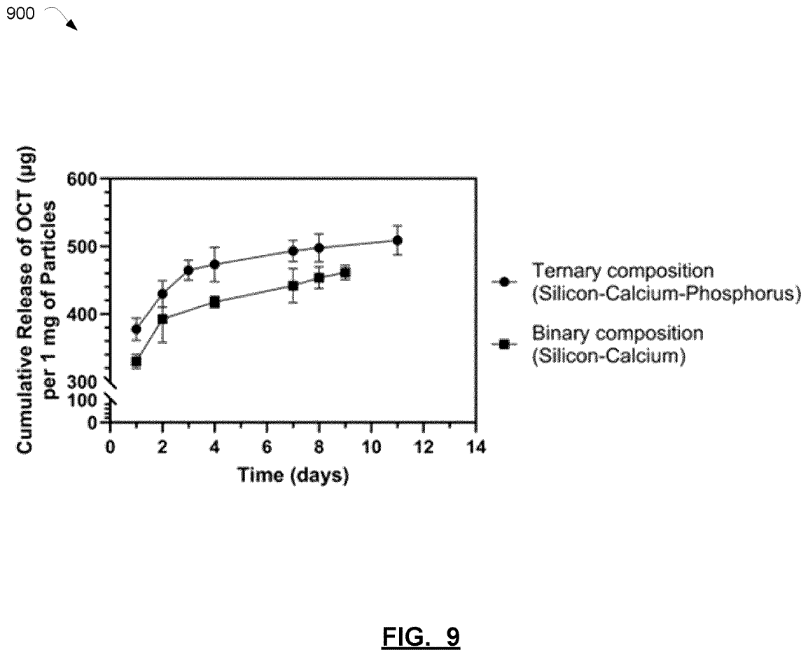

FIG. 9 provides a chart 900 showing the release profile of OCT from a ternary and a binary composition. Both compositions have the same content of CaCl₂, however, the ternary composition containing P₂O₅ showed higher OCT release compared to the binary composition.

While CMC determines the minimum concentration of amphiphilic biologically active agent for micelle formation, the actual biological active agent content in the final material is also influenced by synthesis parameters. For instance, the CMC of octenidine dihydrochloride in water is 3.79 mM; however, changing the synthesis parameters can result in materials with total OCT content varying between 20 to 60 wt %.

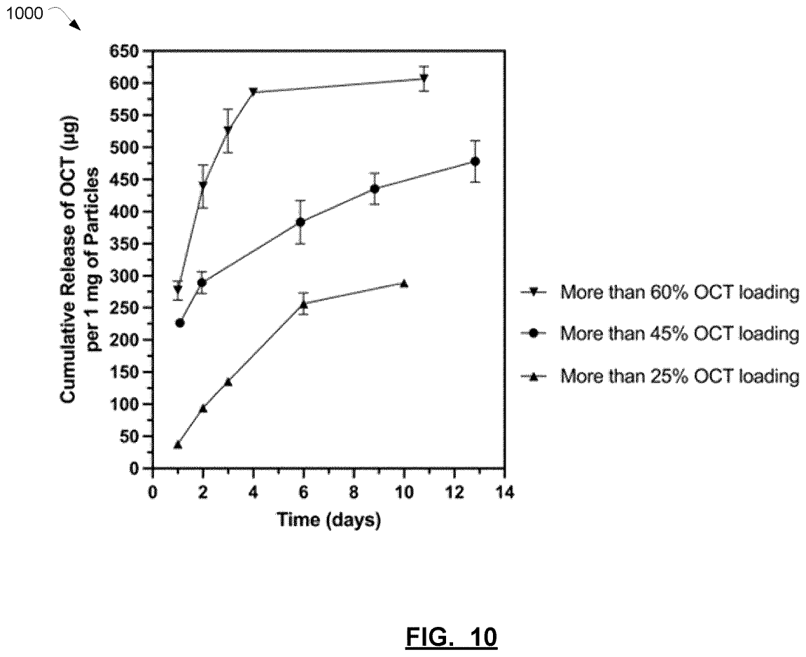

FIG. 10 provides a chart 1000 showing the release profile of OCT from different biocompatible materials with same composition but synthesized under different reaction conditions, including temperature, ionic strength of reaction medium and concentration and timing of addition of precursors. This is in contrast with post-synthesis loading of a compound in the traditional method, in which the loading content is diffusion-limited and cannot be modulated considerably by changing the porous structure and/or pore volume. In the provided examples, the ion(s) included, and the reaction parameters influenced the overall load of biologically active agent (the antimicrobial OCT) in the material, as well as the kinetics of the release of OCT, a useful property of the system when engineering effects for specific applications. The more than 25% OCT loaded particles have a framework of silica. The more than 45% OCT loaded particles have a nominal composition of 85%-15% silica-calcium. The more than 60% OCT loaded particles have a nominal composition of 85%-10%-5% silica-calcium-phosphorous (though these particles were prepared with different synthesis conditions than the particles of FIG. 12 having the same nominal composition). Although the composition varied between these samples, the composition is not believed to be solely responsible for the variations in OCT loading.

Therefore, the biologically active agent content, its release rate and optionally the integrity of the oxide(s) structure can be modulated depending on the requirements of the final application.

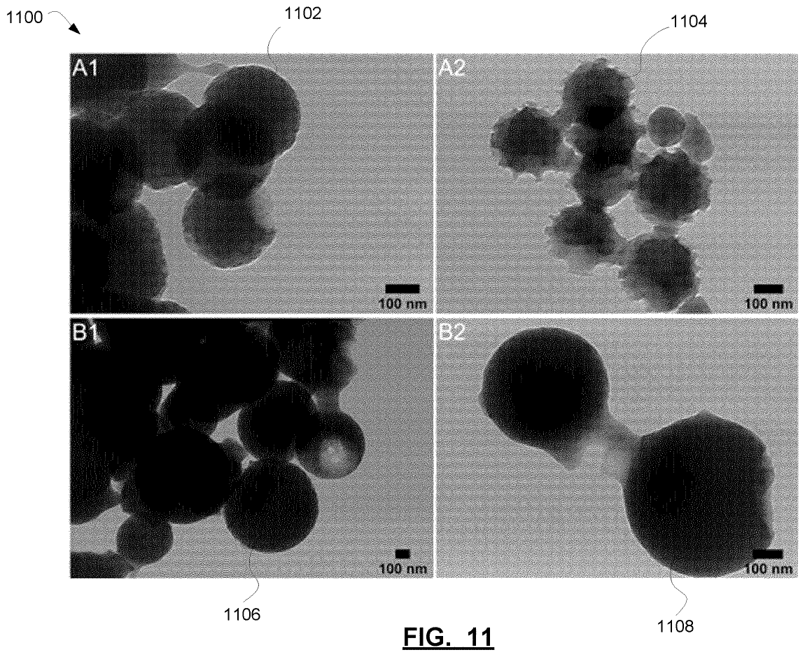

FIG. 11 shows TEM images 1100 of two biocompatible materials after incubation at 37 °C in PBS for 33 days. The oxide structure in images A1 (1102) and A2 (1104) nominally contained 70 mole % SiO₂, while the oxide structure in images B1 (1106) and B2 (1108) nominally contained 90 mole % SiO₂. Release of ions and subsequent degradation/resorption of material can be seen in the images. The material with higher content of SiO₂ showed less degradation/resorption, which can be expected. The degredation/resorption of the particles shown in FIG. 11 may be indicative of a release of ions from the particles but is not believed to indicate degredation of the oxide network. Especially for ions near the surface of the particles, the release of the ions may appear as a degredation/resorption of the particle as a whole even without degredation of the oxide network.

The oxide(s) structure integrity is mainly determined and controlled by its composition and can be modulated depending on the application and with respect to elements included in the network structure. In alterantive embodiments where the porous oxide(s) structure may be considered resorbable or semi-resorbable when exposed to a medium, the release of the biologically active agent does not depend on or result in compromising the oxide(s) structure integrity and any degradation of the oxide structure depends mainly on its composition. Similarly, even in embodiments where the oxide(s) structure may be considered resorbable or semi-resorbable when exposed to a medium, the composition releases the releasable ions independently of any dissolution of the network, i.e. the release of the releasable ions does not require the degradation of the oxide structure although the rate of release could be affected by any optional degredation of the oxide structure. For applications in which the material is used as a filler in a composite and enhancement of the mechanical properties of the composite is one of the purposes that should be served by the material, the ability to preserve the structural integrity, particularly of the oxide(s) structure, despite the release of the biologically active agent is desirable and can be controlled by adjusting the composition of oxide(s) structure. In other applications a partially or completely degradable or resorbable particle or oxide structure may be desired.

FIG. 12 provides a chart 1200 showing the degree of conversion (DC) of polymer resin composites containing different contents of material with a ternary composition (SiO₂—CaCl₂—P₂O₅) nominally containing 85 mole % SiO₂ with 10% calcium and 5% phosphorous.

The particulate material was incorporated into the polymer system through contra-centrifugal mixing with the polymer resin components. However, the described material here may be incorporated in a number of different polymer or material composites through any number of standard techniques used to incorporate solid phases in liquid phases including orbital mixer, shear mixer, sonication, and stirring. This includes but is not limited to those techniques listed here and others used to incorporate glass or solid filler particles and powders into dental materials, such as dental glass into resin composites. Mixing may be performed with an individual component or with a pre-combined combination or optionally a final composition of fillers for the mixture/composite. In addition, the disclosed material is compatible with many organic/inorganic additives for resin composites, including but not limited to polymerization inhibitors and color stabilizers such as 4-Methoxyphenol (MEHQ) or butylated hydroxytoluene (BHT) as organic additives, as well as other inorganic fillers. Octenidine dihydrochloride, optionally included in some embodiments of the composition, includes tertiary amine structures. Some dental resin systems have well known color stability issues related to their photoinitiator systems, especially in the presence of aromatic amines. The addition of MEHQ and/or BHT as color stabilizers in the presence of tertiary or aromatic amines in dental materials is well established and understood, and could be used in resin systems containing the materials disclosed herein. Alternatively, photoinitiator systems may be used that do not have documented color stability problems or do not themselves contain additional tertiary amines, such as those used commercially with the tertiary-amine-containing monomer methacryloyloxydodecylpyridinium bromide (MDPB). Additionally, low trace concentrations of MEHQ, BHT, or other phenolic colour stabilizers may be removed from monomers as received from manufacturers, as low concentrations of these monomers may themselves contribute to discoloration.

In one embodiment, incorporation up to 5 wt % may not affect the DC of resin composite. However, in higher incorporation rates (e.g. 10 wt % and 15 wt %) the DC may decrease. Incorporation at up to or beyond 70% by weight of the biocompatible material can be possible, similar to other solid fillers in composite materials. A deceasing trend for DC with increasing filler content has been reported repeatedly for different types of fillers and is attributed to decreased mobility of reactants or the increased opacity of the resin composite, which reduces the penetration depth of light, rather than due to the presence of ions. Non-ion-releasing material having silica as the sole oxide structure has shown a similar trend in DC.

One optional application of the compositions described herein is as an antimicrobial filler for dental resin composites with a capacity of releasing ions.

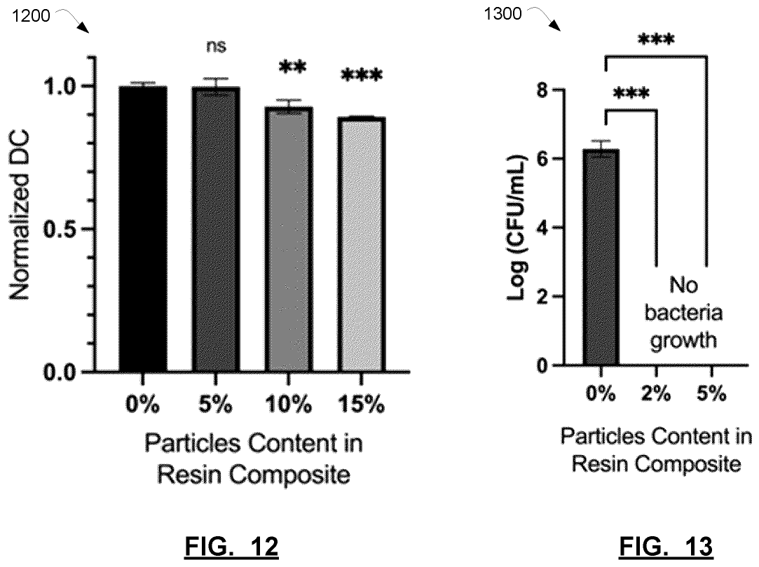

Compared to stand-alone particles, incorporation of particles in a methacrylate-based polymeric matrix (resembling the particles in a potential final application as an antimicrobial dental filler) reduces the release rate of the biologically active agent, possibly due to the increase of diffusion path and/or the hydrophobicity of the matrix. However, this reduced release rate enhances the longevity of the release and consequently, the antimicrobial efficacy of the polymeric matrix containing particles. The reduced rate of release of OCT from particles dispersed in a resin still effectively inhibited the growth of S. mutans in the samples containing 2 wt % or 5 wt % particles compared to the samples without particles, as depicted in FIG. 13.

### EXAMPLES

The following section can provide examples for preparation, use, and synthesis of exemplary compositions.

### Example 1: Preparation of a Binary Composition Comprising a Silicon Dioxide Network Modified with Calcium

In this example, a binary structure was prepared using tetraethyl orthosilicate (TEOS) as a precursor of a network forming compound and calcium chloride as a source of releasable ions. These compounds provide silica and calcium in the composition.

104 mg of octenidine dihydrochloride was added to 14.85 g of water under vigorous stirring. The pH of the solution was increased to 11 using sodium hydroxide (NaOH), which functions as a catalyst. 1.24 mL TEOS was added to the above solution to start the hydrolysis and condensation reactions. After waiting for 5 minutes, 0.27 g of calcium chloride was added to the above solution. The stirring continued for about 30 minutes. The formed solid material was separated, washed twice with water, and dried at about 60 °C.

Particles produced in Example 1 are shown as particles 402, 404 in FIG. 4. They are also the binary composition in FIG. 6A and the binary composition in FIG. 9

### Example 2: Preparation of a Binary Composition Comprising Silicon Dioxide Modified with Phosphorus

In this example, a binary structure was prepared using tetraethyl orthosilicate (TEOS) as a precursor of network forming compound and triethyl phosphate (TEP) as a precursor of a network modifying compound. These compounds provide silica and phosphorous, respectively, in the composition. TEP is believed to act, at least in part, as a precursor for phosphorous pentoxide. Hydrolysis and condensation of TEP results in at least some of the phosphorous being covalently bound to oxygen in the silica framework, thereby forming an SiO₂—P₂O₅ glass. However, some phosphorous may also be present in or on the surface of the framework as non-covalently bound phosphorous or phosphate ions. Optionally, TEP may be considered a precursor of a network forming compound, although it will be referred to as precursor of a network modifying compound in this and following examples since the network is primarily a silicon dioxide network. The TEP may also be considered a source of releasable ions since at least some releasable phosphorous (optionally as phosphate) is believed to be associated with the particles of these examples.

104 mg of octenidine dihydrochloride was added to about 14.85 g of water under vigorous stirring. The pH of the solution was increased to about 11 using sodium hydroxide (NaOH). 1.24 mL TEOS was added to the above solution to start the hydrolysis and condensation reactions followed essentially immediately by the addition of about 0.21 mL TEP. The stirring continued for 30 minutes. The formed solid material was separated, washed twice with water, and dried at about 60 °C.

Particles produced in Example 2 are shown as particles 1106, 1108 in images B1 and B2 of FIG. 11.

### Example 3: Preparation of a Ternary Composition Comprising Silicon Dioxide Modified with Calcium and Phosphorus

In this example, a ternary structure was prepared using tetraethyl orthosilicate (TEOS) as a precursor of a network forming compound, triethyl phosphate (TEP) as a precursor of a network modifying compound and calcium chloride as sources of releasable ions. The TEP may also provide releasable ions. These compounds provide silica, phosphorous and calcium in the composition.

104 mg of octenidine dihydrochloride was added to 14.85 g of water under vigorous stirring. The pH of the solution was increased to about 11 using sodium hydroxide (NaOH). 1.24 mL TEOS was added to the above solution to start the hydrolysis and condensation reactions followed essentially immediately by the addition of 0.124 mL TEP and, after a 5-minute delay, by the addition of 0.269 g of calcium chloride. The stirring continued for about 30 minutes. The formed solid material was separated, washed twice with water, and dried at about 60 °C.

Particles produced in Example 3 are shown as particles 1102,1104 in images A1 and A2 of FIG. 11 and as particles 406, 408 in FIG. 4. These particles are also the ternary composition of silicon-calcium-phosphorous in FIGS. 6A, 6B and 6C, 7, 8, 9 and 13.

In FIG. 6A, the particles of example 3 are described as a ternary composition having a high Ca ions content. The nominal composition of the particles (i.e. the composition based on the amount of reactant added rather than based on analysis of the final product) is 75%-25%-5% (silica-calcium-phosphorous) by mole. The ternary composition having a low Ca ions content is nominally 85%-10%-5% (silica-calcium-phosphorous). The binary composition is nominally 75%-25% (silica-calcium).

In FIG. 8, the particles of example 3 are represented by the dots and have a nominal composition of 75%-25%-5% (silica-calcium-phosphorous). The squares are particles having a nominal composition of 85%-10%-5% (silica-calcium-phosphorous). These particles, having a nominal composition of 85%-10%-5% (silica-calcium-phosphorous), are also described in FIG. 12.

### Example 4: Preparation of a Ternary Composition Comprising Silicon Dioxide Modified with Magnesium and Phosphorus

In this example, a ternary structure was prepared using tetraethyl orthosilicate (TEOS) as a precursor of a network forming compound, triethyl phosphate (TEP) as a precursor of a network forming compound and magnesium chloride as sources of releasable ions. The TEP may also provide releasable ions. These compounds provide silica, phosphorous and magnesium in the composition.

104 mg of octenidine dihydrochloride was added to about 14.85 g of water under vigorous stirring. The pH of the solution was increased to 11 using sodium hydroxide (NaOH). 1.24 mL TEOS was added to the above solution to start the hydrolysis and condensation reactions followed essentially immediately by the addition of 0.11 mL TEP and, after a 5-minute delay, 0.13 g of magnesium chloride. The stirring continued for 30 minutes. The formed solid material was separated, washed twice with water, and dried at about 60 °C.

Particles made according to Example 4 are described in FIG. 6B and FIG. 7.

### Example 5: Preparation of a Ternary Composition Comprising Silicon Dioxide Modified with Potassium and Phosphorus

In this example, a ternary structure was prepared using tetraethyl orthosilicate (TEOS) as a network forming compound, triethyl phosphate (TEP) as a precursor of a network modifying compound and potassium chloride as sources of releasable ions. The TEP may also provide releasable ions. These compounds provide silica, phosphorous and potassium in the composition.

104 mg of octenidine dihydrochloride was added to 14.85 g of water under vigorous stirring. The pH of the solution was increased to 11 using sodium hydroxide (NaOH). About 1.24 mL TEOS was added to the above solution to start the hydrolysis and condensation reactions followed essentially immediately by the addition of 0.11 mL TEP and, after a 5-minute delay, by 0.05 g of potassium chloride. The stirring continued for 30 minutes. The formed solid material was separated, washed twice with water, and dried at about 60 °C.

Particles made according to example 5 are described in FIG. 7.

### Example Applications of the Biologically Active Agent- and Ion(s)-Releasing Materials

### Dental Erosion

Dental erosion refers to the demineralization of the enamel layer from repeated exposure to acidic substances which comes from extrinsic food sources and intrinsic esophageal reflux. The use of calcium fluoride to form a layer covering the enamel can act as a sacrificial layer against dental erosion. This compound is included in many kinds of toothpaste. Calcium ions provide the backbone for hydroxyapatite, while fluoride ions promote the remineralization of calcium and phosphate ions on the surface of teeth. However, this process is regulated by the concentration of both calcium and phosphate ions found in saliva. Compositions described herein that release calcium and/or phosphate ions can be applied, for example in toothpaste, to protect teeth from dental erosion. Optionally, the composition may also release a biologically active agent, for example an antimicrobial agent.

### Dentin Tubule Occlusions

Dentinal hypersensitivity is a painful and common dental problem that is caused by the deterioration of the enamel and exposure of dentin to different stimuli. Dentin contains tubules that link the tooth to the nervous system. The movement of fluids from the exposed parts of the dentin leads to the tubules stimulating the nerve endings, which causes discomfort. Dentin tubule occlusion is a method that is used to treat dentin hypersensitivity. This technique relies on the formation of solid precipitates inside the tubules. Optionally, the tubules may be occluded with calcium-phosphate precipitates. Compositions described herein that release calcium and/or phosphate ions can be used to provide a sustained and long-term release of calcium and phosphate ions to promote occlusion of the tubules. Optionally, the composition may also release a biologically active agent, for example an antimicrobial agent. The composition may be applied to the tubules in a toothpaste composition or in a slurry of particles in water brushed on the surface of the dentin.

### Bone Regeneration

Bone regeneration is a complex problem that relies on balancing the interaction between material and environmental conditions. In bone regeneration, calcium ions are involved in the cellular signaling of the osteoblastic bone synthesis pathway in osteoblasts resulting in the formation of the bone matrix. Phosphorus ions provide the building blocks of key proteins and physiological processes. Compositions as described herein that release calcium and/or phosphorous ions, and optionally a biologically active agent, may be applied in this field. They can be loaded into bone cement and/or scaffolds, depending on the type and extent of bone damage. Once loaded inside, calcium and phosphate ions will slowly release from the material, out of the medical device and into the affected area. The prolonged release of CaP from the material will provide extended and accelerated bone healing. The versatility of the synthesis method described herein allows for additional functional moieties to be encapsulated. Recombinant human bone morphogenic protein-2 (rhBMP-2) is a growth factor that induces osteogenic differentiation and bone formation. To apply a biologically active agent-and ion(s)-releasing material, rhBMP-2 and calcium and phosphate may be loaded into the composition and introduced into existing scaffolding materials. rhBMP-2 may be loaded into particles of a composition after removing the templating agent by combining rhBMP-2 into a solution containing particles of the composition. The mixture is then shaken, for example using a mechanical shaker for 24 hours, to load rhBMP-2 into the particles. rhBMP-2 containing particles can be used in bone scaffolds, cement nanocomposites, or for periodontal tissue regeneration.

### 3D Printing Material

Two general types of 3D printing are: fused deposition modeling (FDM) and stereolithography (SLA). The former melts filaments and solidifies them into the desired structure, while the latter uses lasers to photocure liquid resin into the desired shape. A wide range of biomedical applications is possible as a result of the versatility that 3D printing offers. Medical devices that could potentially be 3D printed include but are not limited to dentures, implants, and bone implants. Compositions as described herein could be applied as an additive to the resin/filament material used in 3D printing. It is expected that compositions as described herein will be compatible with 3D printing resins when their size and shape is tailored to match MSNs previously used as 3D printing additives. Particles of the composition described herein may be added to a 3D printing resin by continuous mixing using a magnetic stir bar followed by sonication using a homogenizer.

### Agriculture

The agricultural field has benefited from nanoparticles as they have been shown to help detect and manage plant pathogens, control pesticides and plant diseases, and also increase tolerance to biotic and abiotic stresses. Compositions as described herein can be synthesized with a variety of small molecule biologically active payloads that are useful in the agricultural industry. For examples, avermectin or pyoluteorin may be encapsulated into silica nanoparticles through a high-gravity reactive precipitation method. Urea may be encapsulated by incubating different w/v of urea with a suspension of MSN for an extended period of time.

### Cosmetics

In the field of cosmetics, the skin is an important organ that needs to be penetrated by cosmetic ingredients for some effects to be realized. Compositions as described herein may be loaded with, for example, retinol, quercetin or octyl methoxycinnamate (OMC).

While the invention has been illustrated and described with reference to preferred embodiments thereof, it will be recognized by those skilled in the art that various changes in form and detail may be made therein without departing from the spirit and scope of the invention as defined by the appended claims.

1. A composition comprising
a porous oxide network;
one or more releasable ions within the network and/or on the surface of the network, wherein the one or more releasable ions are not covalently bound to the network and/or the composition releases the one or more releasable ions independently of any dissolution of the network; and,
one or more biologically active agents contained in pores of the network.

2. The composition of claim 1 wherein the one or more biologically active agents comprises an amphiphilic templating agent present in a self-assembled or micellar form.

3. The composition of claim 1 wherein the network comprises one or more metals covalently bound to oxygen, wherein the one or more metals optionally includes a metalloid, for example silicon.

4. The composition of claim 3 wherein the network further comprises one or more non-metals covalently bound to oxygen, optionally wherein the network comprises a silica-phosphorous-oxide.

5. The composition of claim 1 wherein the one or more releasable ions comprise one or more monoatomic ions, optionally one or more of calcium, phosphorous, magnesium and potassium.

6. The compositions of claim 1 wherein the one or more releasable ions comprise a polyatomic ion, for example phosphate.

7. The composition of claim 1 wherein the one or more biologically active agents comprises an antimicrobial agent.

8. The composition of claim 1 wherein the biologically active agent is any one of octenidine dihydrochloride, polyhexamethylene biguanide, cetylpyridinium chloride, lauric arginate, and benzalkonium chloride.

9. The composition of claim 1 comprising a silica-oxide or silica-phosphorous-oxide network, an antimicrobial agent in the pores of the network, and one or more of calcium, phosphorous, magnesium, potassium and phosphate ions associated with the network.

10. A composite material comprising the composition of claim 1 dispersed in a matrix material.

11. The composite material of claim 10 wherein the matrix material is a polymeric resin, for example a dental adhesive or restorative resin.

12. The composite material of claim 10 formulated for use as a dental material, an orthopedic implant or bone cement material, a 3D printing ink or filament material, a cosmetic material, or an agricultural material.

13. A method of making a composition comprising,
preparing a solution of one or more templating agents, optionally with a catalyst;
adding one or more precursors of one or more metal oxide network forming compounds to the solution;
adding a) one or more precursors of one or more non-metal network modifying compounds or elements or b) one or more sources of releasable ions, or both a) and b), to the solution; and,
allowing a reaction to proceed whereby a solid material condenses around the templating agent, wherein the solid material comprises a porous oxide network with one or more releasable ions within the network and/or on the surface of the network.

14. The method of claim 13 wherein the one or more sources of releasable ions are added to the solution at least 1 minute, at least 3 minutes or at least 5 minutes after adding the one or more precursors of one or more metal oxide network forming compounds to the solution.

15. The method of claim 13 wherein the one or more precursors of one or more metal oxide network forming compounds comprise tetraethyl orthosilicate (TEOS), the one or more precursors of one or more non-metal network modifying compounds or elements comprise triethyl phosphate (TEP) and the one or more one or more sources of releasable ions comprise one or more metal salts, optionally calcium chloride, potassium chloride and/or magnesium chloride.

16. The method of claim 13 wherein the one or more templating agents comprise a biologically active templating agent.

17. The method of claim 13 wherein the one or more templating agents comprise an antimicrobial agent, for example OCT.

18. The method of claim 13 further comprising mixing the particles in a matrix material.

19. The method of claim 18 wherein the matrix material is a polymeric resin, for example a dental adhesive or restorative resin.

20. The method of claim 18 wherein the particles are incorporated into the matrix material by one or more of contra-centrifugal mixing, orbital mixing, shear mixing, sonication, and stirring with polymer resin components.

21-85. (canceled)
